(19) United States
(12) Patent Application Publication
(10) Pub. No.: US 2015/0299621 A1
(43) Pub. Date: Oct. 22, 2015

(54) FORMULATION FOR CLEANSER FOR AUTOCLAVE, FRUITS & VEGETABLES, PEST CONTROL, & SHAMPOO

(71) Applicants: Daniel Peretz, New York, NY (US); David Levy, Deal, NJ (US)

(72) Inventors: DANIEL PERETZ, New York, NY (US); David Levy, Deal, NJ (US)

(21) Appl. No.: 14/258,548

(22) Filed: Apr. 22, 2014

(51) Int. Cl. C11D 3/382; A61K 8/97; A61Q 5/02; C11D 11/00; A01N 25/02

(52) U.S. Cl. CPC C11D 3/382; C11D 11/0041; C11D 11/0017; A01N 25/02; A61Q 5/02; A61K 8/97

(57) ABSTRACT

A cleaner for an autoclave unit, comprising citric acid having a range of 30 to 80 pounds; mineral water having a range of 23 to 53 gallons; natural lemon juice having a range of ½ to 3 gallons; cloves having a range of ½ to 3 gallons; garlic having a range of ½ to 3 gallons; peppermint having a range of ½ to 3 gallons; thyme having a range of ½ to 3 gallons; cinnamon having a range of ½ to 3 gallons; pimento having a range of ½ to 3 gallons; and a bio-detergent powder mix having a range of 4 to 10 gallons. The invention also includes a cleaner for fruits and vegetables, a pest control composition, and a shampoo composition.

### FIELD OF THE INVENTION

The present invention relates to novel cleaning formulations, each having a unique combination of ingredients to provide a natural and optimum cleanser.

### BACKGROUND OF THE INVENTION

Presently existing cleansers do not meet the goal of providing an optimum cleanser using natural ingredients. The present invention meets this goal since the ingredients provide a unique combination, and the quantities of the ingredients are also a unique combination. The details are set forth below.

### OBJECTS OF THE INVENTION

It is an object of the present invention to provide a unique combination of ingredients and a unique combination of quantities of each ingredient, which provide an optimum cleanser using natural ingredients.

### SUMMARY OF THE INVENTION

The present invention provides the following unique combinations of ingredients and quantities for cleaning for the following 4 embodiments: an autoclave cleanser, a cleanser for fruits and vegetables, a pest control formulation, and an organic shampoo.

### First Embodiment

### Autoclave Cleanser (50 Gallons)

The ingredients are:

Citric Acid (50 pounds)
Mineral Water (38 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Bio Detergent Powder Mix (5 gallons)
Total - 50 gallons

### Second Embodiment

### Fruit and Vegetable Cleanser (50 Gallons)

The ingredients are:

Wassabe Vinegar (3 gallons)
Lactic Acid (24 ounces)
Mineral Water (34.75 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Cedar Oil (1 liter);
Coconut Oil (1 liter);
Anise Oil (1 liter);
Lactic Acid (1 liter);
Bio Detergent Powder Mix (Ecological Soap) (5 gallons)
Total - 50 gallons

### Third Embodiment

### Pest Control (50 Gallons)

The ingredients are:

Brewers Yeast (powder)
Wassabe Vinegar (3 gallons)
Lactic Acid (24 ounces)
Mineral Water (34.75 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Cedar Oil (1 liter);
Coconut Oil (1 liter);
Anise Oil (1 liter);
Lactic Acid (1 liter);
Bio Detergent Powder Mix (Ecological Soap) (5 gallons)
Total - 50 gallons

### Fourth Embodiment

### Shampoo (2 Gallons)

The ingredients are:

Brewers Yeast (powder)
Mineral Water (1 gallon plus 89 ounces) (the other ingredients
add up to 39 oz.)
Microbial Mix (7 ingredients - 35 ounces - 5 ounces each)
Natural Lemon Juice (5 ounces)
Cloves (5 ounces)
Garlic (5 ounces)
Peppermint (5 ounces)
Thyme (5 ounces)
Cinnamon (5 ounces)
Pimento (5 ounces)
(All 7 are heated to 140°)
Cedar Oil (1 ounce)
Coconut Oil (1 ounce)
Anise Oil (1 ounce)
Lactic Acid (¾ ounce)
Bio Detergent Powder Mix (powder)
Total - 2 gallons

### DETAILED DESCRIPTION OF THE PREFERRED EMBODIMENT

The present invention provides the following unique combinations of ingredients and quantities for cleaning for the following 8 embodiments: an autoclave cleanser, a cleanser for fruits and vegetables, a cleanser for pest control, and a shampoo cleanser.

### First Embodiment

### Autoclave Cleanser (50 Gallons)

This is an organic and natural liquid cleaner for an autoclave. The ingredients are:

Citric Acid (50 pounds)
Mineral Water (38 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Bio Detergent Powder Mix (5 gallons)
Total - 50 gallons

### Second Embodiment

### Organic Fruit and Vegetable Cleanser (50 Gallons)

This is an organic and natural cleaner for fruits and vegetables to remove insects, worms, and microbes. The ingredients are:

Wassabe Vinegar (3 gallons)
Lactic Acid (24 ounces)
Mineral Water (34.75 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Cedar Oil (1 liter)
Coconut Oil (1 liter)
Anise Oil (1 liter)
Lactic Acid (1 liter)
Bio Detergent Powder Mix (Ecological Soap) (5 gallons)
Total - 50 gallons

### Third Embodiment

### Organic Pest Control (50 Gallons)

This is an organic and natural pest control that removes and kills fleas, bugs, and contaminants. The ingredients are:

Brewer's Yeast (powder)
Wassabe Vinegar (3 gallons)
Lactic Acid (.25 gallons)
Mineral Water (34.75 gallons)
Microbial Mix (7 ingredients - 7 gallons)
Natural Lemon Juice (1 gallon)
Cloves (1 gallon)
Garlic (1 gallon)
Peppermint (1 gallon)
Thyme (1 gallon)
Cinnamon (1 gallon)
Pimento (1 gallon)
(All 7 are heated to 140°)
Cedar Oil (1 liter)
Coconut Oil (1 liter)
Anise Oil (1 liter)
Lactic Acid (1 liter)
Bio Detergent Powder Mix (Ecological Soap)
(5 gallons) - Total - 50 gallons

### Fourth Embodiment

### Organic Shampoo (2 Gallons)

This is an organic and natural shampoo that removes and kills ticks, lice, and contaminants. The ingredients are:

Brewer's Yeast (powder)
Mineral Water (1 gallon plus 89 ounces) (the other ingredients
add up to 39 oz.)
Microbial Mix (7 ingredients - 35 ounces - 5 ounces each)
Natural Lemon Juice (5 ounces)
Cloves (5 ounces)
Garlic (5 ounces)
Peppermint (5 ounces)
Thyme (5 ounces)
Cinnamon (5 ounces)
Pimento (5 ounces)
(All 7 are heated to 140°)
Cedar Oil (1 ounce)
Coconut Oil (1 ounce)
Anise Oil (1 ounce)
Lactic Acid (¾ ounce)
Bio Detergent Powder Mix (powder)
Total - 2 gallons

### Fifth Embodiment (Ranges)

### Autoclave Cleanser (50 Gallons)

This is an organic and natural liquid cleaner for an autoclave. The ingredients are:

Citric Acid (30 to 80 pounds)
Mineral Water (23 to 53 gallons)
Microbial Mix (7 ingredients)
Natural Lemon Juice (½ to 3 gallons)
Cloves (½ to 3 gallons)
Garlic (½ to 3 gallons)
Peppermint (½ to 3 gallons)
Thyme (½ to 3 gallons)
Cinnamon (½ to 3 gallons)
Pimento (½ to 3 gallons)
(All 7 are heated to 140°)
Bio Detergent Powder Mix (4 to 10 gallons)
Total - 50 gallons

### Sixth Embodiment (Ranges)

### Organic Fruit and Vegetable Cleanser (50 Gallons)

This is an organic and natural cleaner for fruits and vegetables to remove insects, worms, and microbes. The ingredients are:

Wassabe Vinegar (2 to 4 gallons)
Lactic Acid (24 to 40 ounces)
Mineral Water (24.75 to 44.75 gallons)
Microbial Mix (7 ingredients)
Natural Lemon Juice (½ to 3 gallons)
Cloves (½ to 3 gallons)
Garlic (½ to 3 gallons)
Peppermint (½ to 3 gallons)
Thyme (½ to 3 gallons)
Cinnamon (½ to 3 gallons)
Pimento (½ to 3 gallons)
(All 7 are heated to 140°)
Cedar Oil (1 to 1.5 liters)
Coconut Oil (1 to 1.5 liters)
Anise Oil (1 to 1.5 liters)
Lactic Acid (1 to 1.5 liters)
Bio Detergent Powder Mix (Ecological Soap) (4 to 10 gallons)
Total - 50 gallons

### Seventh Embodiment (Ranges)

### Organic Pest Control (50 Gallons)

This is an organic and natural pest control that removes and kills fleas, bugs, and contaminants. The ingredients are:

Brewers Yeast (powder)
Wassabe Vinegar (2 to 4 gallons)
Lactic Acid (24 to 40 ounces)
Mineral Water (24.75 to 44.75 gallons)
Microbial Mix (7 ingredients - 7 gallons)
Natural Lemon Juice (½ to 3 gallons)
Cloves (½ to 3 gallons)
Garlic (½ to 3 gallons)
Peppermint (½ to 3 gallons)
Thyme (½ to 3 gallons)
Cinnamon (½ to 3 gallons)
Pimento (½ to 3 gallons)
(All 7 are heated to 140°)
Cedar Oil (1 to 1.5 liters)
Coconut Oil (1 to 1.5 liters)
Anise Oil (1 to 1.5 liters)
Lactic Acid (1 to 1.5 liters)
Bio Detergent Powder Mix (Ecological Soap)
(4 to 10 gallons) - Total - 50 gallons

### Eighth Embodiment (Ranges)

### Organic Shampoo (2 Gallons)

This is an organic and natural shampoo that removes and kills ticks, lice, and contaminants. The ingredients are:

Brewers Yeast (powder)
Mineral Water (1.25 gallons to 3.0 gallons)
Microbial Mix (7 ingredients)
Natural Lemon Juice (3 to 8 ounces)
Cloves (3 to 8 ounces)
Garlic (3 to 8 ounces)
Peppermint (3 to 8 ounces)
Thyme (3 to 8 ounces)
Cinnamon (3 to 8 ounces)
Pimento (3 to 8 ounces)
(All 7 are heated to 140°)
Cedar Oil (¾ to 2 ounces)
Coconut Oil (¾ to 2 ounces)
Anise Oil (¾ to 2 ounces)
Lactic Acid (¾ to 2 ounces)
Bio Detergent Powder Mix (powder)
Total - 2 gallons

### ADVANTAGES OF THE PRESENT INVENTION

It is an advantage of the present invention to provide a unique combination of ingredients and a unique combination of quantities of each ingredient, which provide optimum cleansers, using natural ingredients.

A latitude of modification, change and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1) A cleaner for an autoclave unit, comprising:
(a) citric acid having a range of 30 to 80 pounds;
(b) mineral water having a range of 23 to 53 gallons;
(c) natural lemon juice having a range of ½ to 3 gallons;
(d) cloves having a range of ½ to 3 gallons;
(e) garlic having a range of ½ to 3 gallons;
(f) peppermint having a range of ½ to 3 gallons;
(g) thyme having a range of ½ to 3 gallons;
(h) cinnamon having a range of ½ to 3 gallons;
(i) pimento having a range of ½ to 3 gallons; and
(j) a bio-detergent powder mix having a range of 4 to 10 gallons.

2) A cleaner for fruits and vegetables, comprising:
(a) wasabe vinegar having a range of 2 to 4 gallons:
(b) lactic acid having a range of 24 to 40 ounces;
(c) mineral water having a range of 24.75 to 44.75 gallons;
(d) natural lemon having a range of ½ to 3 gallons;
(e) cloves having a range of ½ to 3 gallons;
(f) garlic having a range of ½ to 3 gallons;
(g) peppermint having a range of ½ to 3 gallons;
(h) thyme having a range of ½ to 3 gallons:.
(i) cinnamon having a range of ½ to 3 gallons;
(j) pimento having a range of ½ to 3 gallons;
(k) Cedar Oil (1 to 1.5 liters);
(l) Coconut Oil (1 to 1.5 liters);
(m) Anise Oil (1 to 1.5 liters);
(n) Lactic Acid (1 to 1.5 liters; and
(o) bio-detergent powder mix (organic soap) having a range of 4 to 10 gallons;

3) A pest control composition, comprising:
(a) brewer's yeast in the form of powder;
(b) wasabe vinegar having a range of 2 to 4 gallons;
(c) lactic acid having a range of 24 to 40 ounces;
(d) mineral water having a range of 24.75 to 44.75 gallons;
(e) natural lemon having a range of ½ to 3 gallons;
(f) cloves having a range of ½ to 3 gallons;
(g) garlic having a range of ½ to 3 gallons;
(h) peppermint having a range of ½ to 3 gallons;
(i) thyme having a range of ½ to 3 gallons;
(j) cinnamon having a range of ½ to 3 gallons;
(k) pimento having a range of ½ to 3 gallons;
(l) Cedar Oil (1 to 1.5 liters);
(m) Coconut Oil (1 to 1.5 liters);
(n) Anise Oil (1 to 1.5 liters);
(o) Lactic Acid (1 to 1.5 liters; and
(p) bio-detergent powder mix (organic soap) having a range of 4 to 10 gallons;

4) A shampoo for animals and humans, comprising:
(a) brewer's yeast in the form of powder;
(b) mineral water having a range of 1.25 to 3 gallons;
(c) natural lemon juice having a range of 3 to 8 ounces;
(d) cloves having a range of 3 to 8 ounces;
(e) garlic having a range of 3 to 8 ounces;
(f) peppermint having a range of 3 to 8 ounces;
(g) thyme having a range of 3 to 8 ounces;
(h) cinnamon having a range of 3 to 8 ounces;
(i) pimento having a range of 3 to 8 ounces;
(j) cedar oil in the range of ¾ to 2 ounces;
(k) coconut oil in the range of ¾ to 2 ounces;
(l) anise oil in the range of ¾ to 2 ounces;
(m) lactic acid in the range of ¾ to 2 ounces; and
(n) bio detergent powder mix in the form of a powder.

5) A cleaner for an autoclave unit, comprising:
(a) citric acid in the amount of 50 pounds;
(b) mineral water in the amount of 38 gallons;
(c) natural lemon juice in the amount of 1 gallon;
(d) cloves in the amount of 1 gallon;
(e) garlic in the amount of 1 gallon;
(f) peppermint in the amount of 1 gallon;
(g) thyme in the amount of 1 gallon;
(h) cinnamon in the amount of 1 gallon;
(i) pimento in the amount of 1 gallon; and
(j) bio detergent powder mix in the amount of 5 gallons.

6) A cleaner for fruits and vegetables, comprising:
(a) wassabe vinegar in the amount of 3 gallons;
(b) lactic acid in the amount of 24 ounces;
(c) mineral water in the amount of 34.75 gallons;
(d) natural lemon juice in the amount of 1 gallon;
(e) cloves in the amount of 1 gallon;
(f) garlic in the amount of 1 gallon;
(g) peppermint in the amount of 1 gallon;
(h) thyme in the amount of 1 gallon;
(i) cinnamon in the amount of 1 gallon;
(j) pimento in the amount of 1 gallon;
(k) Cedar Oil (1 liter);
(l) Coconut Oil (1 liter);
(m) Anise Oil (1 liter);
(n) Lactic Acid (1 liter); and
(o) bio detergent powder mix (Ecological Soap) in the amount of 5 gallons.

7) A pest control composition, comprising:
(a) brewer's yeast in the form of a powder;
(b) wassabe vinegar in the amount of 3 gallons;
(c) lactic acid in the amount of 24 ounces;
(d) mineral water in the amount of 34.75 gallons;
(e) natural lemon juice in the amount of 1 gallon;
(f) cloves in the amount of 1 gallon;
(g) garlic in the amount of 1 gallon;
(h) peppermint in the amount of 1 gallon;
(i) thyme in the amount of 1 gallon;
(j) cinnamon in the amount of 1 gallon;
(k) pimento in the amount of 1 gallon;
(l) Cedar Oil (1 liter);
(m) Coconut Oil (1 liter);
(n) Anise Oil (1 liter);
(o) Lactic Acid (1 liter); and
(p) bio detergent powder mix (Ecological Soap) in the amount of 5 gallons.

8) A shampoo for animals and humans, comprising:
(a) brewer's yeast in the form of a powder;
(b) mineral water in the amount of 1 gallon plus 89 ounces;
(c) natural lemon juice in the amount of 5 ounces;
(d) cloves in the amount of 5 ounces;
(e) garlic in the amount of 5 ounces;
(f) peppermint in the amount of 5 ounces;
(g) thyme in the amount of 5 ounces;
(h) cinnamon in the amount of 5 ounces;
(i) pimento in the amount of 5 ounces;
(j) cedar oil in the amount of 1 ounce;
(k) coconut oil in the amount of 1 ounce;
(l) anise oil in the amount of 1 ounce;
(m) lactic Acid in the amount of ¾ ounce; and
(n) bio detergent powder mix in the form of a powder.
